(19) United States
(12) Patent Application Publication
(10) Pub. No.: US 2018/0169061 A1
(43) Pub. Date: Jun. 21, 2018

(54) NON-AQUEOUS DELTA9-TETRAHYDROCANNABINOL ORAL LIQUID FORMULATIONS

(71) Applicant: Ascent Pharmaceuticals, Inc., Hauppauge, NY (US)

(72) Inventors: Sridhar Gumudavelli, Hauppauge, NY (US); Madhava Reddy, Hauppauge, NY (US); Sudhakar Vidiyala, Hauppauge, NY (US)

(21) Appl. No.: 15/838,973

(22) Filed: Dec. 12, 2017

Related U.S. Application Data
(60) Provisional application No. 62/434,482, filed on Dec. 15, 2016.

(51) Int. Cl. A61K 31/352; A61K 47/10; A61K 47/14; A61K 47/22; A61K 47/26; A61K 47/44; A61K 9/107; A61K 9/50; A61P 1/08

(52) U.S. Cl. CPC A61K 31/352; A61K 47/10; A61K 47/14; A61K 47/22; A61P 1/08; A61K 47/44; A61K 9/1075; A61K 9/50; A61K 47/26

(57) ABSTRACT

The present invention discloses a means to improve stability, dissolution, and in vivo bioavailability of non-aqueous oral formulations containing natural or synthetic cannabinoid derivatives. In particular, this invention provides a non-aqueous cannabinoid formulation containing zero amount of water with one or more organic solvents, surfactants, co-surfactants, semi-polar solvents and mono-di glycerides. Upon administration, these formulations produce nano-sized cannabinoid liquid globules.

This application claims benefit of U.S. Provisional Application No. 62/434,482 filed on Dec. 15, 2016, the entirety of which is incorporated herein by reference.

### FIELD OF THE INVENTION

The present invention relates to a non-aqueous oral cannabinoid solution suitable for oral administration comprising one or more organic solvents, surfactants, co-surfactants, semi-polar solvents and mono-di glycerides. The present invention offers improved stability, dissolution and bioavailability and can be administered as either a liquid or encapsulated in soft gelatin or hard gelatin capsules form for indications such as nausea and vomiting in chemotherapy and for appetite stimulation in AIDS patients.

### BACKGROUND OF THE INVENTION

Cannabinoids have been employed in the treatment of common symptoms associated with pre-existing or acquired medical conditions such as analgesia, emesis and anorexia. FDA has formally approved the use of Dronabinol in dosage forms such as soft gelatin capsules and more recently an aqueous based oral liquid. The present invention provides therapeutic non-aqueous oral liquid formulation with improved stability, dissolution and bioavailability of Cannabinoid and its derivatives, especially Δ⁹-Tetrahydrocannabinol (Dronabinol).

In particular, the present invention improves stability by protecting the drug from gastrointestinal degradation, improves safety by preventing dumping of the drug in the gastrointestinal tract, and improves bioavailability with or without self-emulsification process by interaction with gastrointestinal tract contents. The present invention also boasts of an improved stability at room and elevated temperatures.

All references cited herein are incorporated herein by reference in their entireties.

### BRIEF SUMMARY OF THE INVENTION

The present invention is related to a non-aqueous, oral solution containing cannabinoids dissolved in nonpolar or semipolar solvents along with other functional and non-functional ingredients including absorption modifiers such as mono-di glycerides, surfactants, co-surfactants and additionally anti-oxidants and preservatives may be added to the final formulation to enhance the stability of the product.

The present invention provides a non-aqueous medium for the dissolution of the active ingredient that protects the drug from dumping in the gastrointestinal region thus preventing the acid degradation of the drug as well as undesired plasma concentrations. The absorption modifiers employed such as mono-di glycerides, surfactants and co-surfactants act in a way to enhance lymphatic transport of the drug due to the formation of chylomicrons that carry the drug to the desired region reliably and thus avoids extensive first pass metabolism that might occur otherwise.

The present invention also describes the formation of micro or nanoemulsions for enhanced bioavailability due to the interaction of the non-aqueous medium containing at least one organic polar or nonpolar solvent with gastrointestinal fluids that are predominantly aqueous buffers either due to the presence of surfactants or cosurfactant agents or due to the presence of mono-di glycerides.

The present invention describes a formulation containing dronabinol dissolved in a non-aqueous medium comprising of 15-50% w/w of dehydrated alcohol, 0-80% w/w of polyfunctional organic entities including polyalkylated glycols such as polyethylene glycol, propylene glycol that act as solvents and co-solvents, along with absorption modulators comprising of 0-50% w/w of surfactant and co-surfactant groups including esterified mixtures of polyalkylene polyols and fatty acids such as PEG-8 caprylic/capryic glycerides (LabrafilTM or Labrasol TM), polyoxyethylene glycolated castor oils such as polyoxyl 35 castor oil or polyoxy 40 hydrogenated castor oil, stability enhancing agents containing antioxidants such as Tocopherol, Butylated hydroxytoluene, butylated hydroxyanisole, propylgallate used alone or as a mixture.

The invention provides an oral non-aqueous formulation comprising: at least one active cannabinoid; at least one organic solvent selected from the group consisting of dehydrated alcohol, polyhydric alcohols, polyethylene glycol and propylene glycol, and mixtures thereof; at least one absorption modifier selected from the group consisting mono-di glycerides, esterified polyalkylene polyols, polyoxyethylene glycolated oils, polyoxyethylene glycolated castor oil, and combinations thereof; at least one stability enhancing agent selected from the group consisting of tocopherol, butylated hydroxytoluene, butylated hydroxyl anisole, and combinations thereof; at least one sweetening agents selected from the group consisting of Sucralose, Saccharin Sodium, ethyl maltol, and other flavoring agents. The invention provides an oral non-aqueous formulation wherein the active cannabinoid is Dronabinol or Δ⁹-Tetrahydrocannabinol. The invention provides an oral non-aqueous formulation wherein the composition comprises a mixture of solvents and the mixture of organic solvents constitutes 50-95% w/w of the formulation. The invention provides an oral non-aqueous formulation wherein the organic solvent is dehydrated alcohol and constitutes 15-50% w/w of the formulation. The invention provides an oral non-aqueous formulation wherein the organic solvent is a polyhydric alcohol selected from the group consisting of sorbitol and glycerol, and constitutes 0-30% w/w of the formulation. The invention provides an oral non-aqueous formulation wherein the organic solvent is polyethylene glycol which is of a lower molecular weight ranging from 100-800 g/mol and constitutes 5-60% w/w of the entire formulation. The invention provides an oral non-aqueous formulation wherein the organic solvent is propylene glycol and constitutes 5-45% w/w of the entire formulation. The invention provides an oral non-aqueous formulation wherein the absorption modulator is polyoxyethylene glycolated castor oil selected from the group consisting of polyoxy 40 hydrogenated castor oil and constitutes 20-40% w/w of the entire formulation. The invention provides an oral non-aqueous formulation wherein the absorption modulator is a mixture of polyoxyethylene glycolated castor oils and PEGlyated glycerides of fatty acids, and comprises of 0-50% w/w of the entire formulation. The invention provides an oral non-aqueous formulation wherein the absorption modulator comprises a mixture of polyoxyethylene glycolated castor oil which is polyoxy 40 hydrogenated castor oil, and the PEGlyated glycerides of fatty acid is −8 caprylic/capric glycerides (LabrafilTM or LabrasolTM). The invention provides an oral non-aqueous formulation wherein the stability enhancing agent is selected from the group consisting of Tocopherol, BHA, BHT and mixtures thereof, in which comprises 0-2% w/w of the entire formulation. The invention provides an oral non-aqueous formulation wherein the sweetening agent is selected from the group consisting of sucralose, saccharin sodium, ethyl maltol, and mixtures thereof and constitutes of about 0-10% w/w of the formulation.

The invention provides a method of treating a patient for a condition selected from the group consisting of pain, emesis, and anorexia comprising: selecting a patient in need of such treatment; administering the composition of the invention, wherein the patient is treated.

### DETAILED DESCRIPTION OF THE INVENTION

As used herein, the term “pharmaceutically acceptable salts” includes acid addition salts or addition salts of free bases. The term “pharmaceutically acceptable salts” of a compound of the invention is also meant to include within its scope all the possible isomers and their mixtures, and any pharmaceutically acceptable metabolite, bioprecursor and/or pro-drug, such as, for example, a compound which has a structural formula different from the one of the compounds of the invention, and yet is directly or indirectly converted in vivo into a compound of the invention, upon administration to a subject, such as a mammal, particularly a human being.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the term “agent” refers to any molecule, compound, methodology and/or substance for use in the prevention, treatment, management and/or diagnosis of a disease or condition. As used herein, the term “effective amount” refers to the amount of a therapy that is sufficient to result in the prevention of the development, recurrence, or onset of a disease or condition, and one or more symptoms thereof, to enhance or improve the prophylactic effect(s) of another therapy, reduce the severity, the duration of a disease or condition, ameliorate one or more symptoms of a disease or condition, prevent the advancement of a disease or condition, cause regression of a disease or condition, and/or enhance or improve the therapeutic effect(s) of another therapy.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “therapeutic agent” refers to any molecule, compound, and/or substance that is used for the purpose of treating and/or managing a disease or disorder.

As used herein, the terms “therapies” and “therapy” can refer to any method(s), composition(s), and/or agent(s) that can be used in the prevention, treatment and/or management of a disease or condition, or one or more symptoms thereof. In certain embodiments, the terms “therapy” and “therapies” refer to small molecule therapy.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, such as pain, emesis, and anorexia and/or the amelioration of one or more symptoms thereof resulting from the administration of one or more therapies. In certain embodiments, the treatment includes a step of selecting a patient in need of treatment.

The term “derivative” or “derivatized” as used herein includes chemical modification of a compound of the invention, or pharmaceutically acceptable salts thereof or mixtures thereof. That is, a “derivative” may be a functional equivalent of a compound of the invention, which is capable of inducing the improved pharmacological functional activity in a given subject. As used herein, the terms “composition” and “formulation” are used interchangeably.

In accordance with certain embodiments, it is an object of the invention to provide a method of controlling nausea and vomiting associated with a human receiving chemotherapy comprising the oral administration of a non-aqueous oral formulation to a human patient experiencing nausea and vomiting, said non-aqueous oral formulation comprising an effective amount of a cannabinoid dispersed in a pharmaceutically acceptable carrier. Preferably, the cannabinoid is dronabinol.

In accordance with certain embodiments, it is a further object of the invention to provide a method of appetite stimulation of an AIDS patient suffering from wasting syndrome comprising the oral administration of a non-aqueous oral formulation to a human patient experiencing a lack of appetite, said non-aqueous oral formulation comprising an effective amount of a cannabinoid dispersed in a pharmaceutically acceptable carrier. Preferably, the cannabinoid is dronabinol.

### Formulations

Cannabinoids in general, and dronabinol specifically, are insoluble in water. The formulations of the present invention therefore preferably include one or more pharmaceutically acceptable organic cosolvents for the cannabinoid. The organic cosolvent will be present in an amount effective to have the cannabinoid substantially solubilized in the organic cosolvent. Therefore, the amount of organic solvent in the formulation will vary based on the concentration of the cannabinoid. The amount of organic cosolvent will also vary based on the partition coefficient of the particular cannabinoid molecule.

### Organic Solvents

A non-aqueous solvent is an organic solvent that is substantially free of water. In certain embodiments, the organic solvent may be selected from, for example, ethanol, propanol, isopropanol, propylene glycol, polyethylene glycol, and combinations thereof that are pharmaceutically acceptable based on the intended route of administration of the desired formulation. Suitable organic solvents may include, but are not limited to, acetonitrile, chlorobenzene, chloroform, cyclohexane, 1,2-dichlorethane, dichloromethane, 1,2-dimethoxyethane, N,N,-dimethylacetamide, N,N-dimethylformamide, 1,4 dioxane, 2-ethoxyethanol, ethylene glycol, formamide, hexanes, 2-methoxyethanol, methyl butyl ketone, methylcyclohexane, N-methyl-pyrrolidone, nitromethane, pyridine, sulfolane, tetralin, toluene, 1,2-trichlorethane, xylene, acetic acid, acetone, anisole, butyl acetate, tert-butyl ethyl ether, cumene, dimethylsulfoxide, ethyl acetate, ethyl ether, ethyl formate, formic acid, heptanes, isobutyl acetate, isopropyl acetate, isopropyl alcohol, methyl acetate, methyl ethyl ketone, methyl isobutyl ketone, pentanel, propyl acetate, tetrahydrofuran, C₁-C₆ alcohols, and any mixtures of two or more thereof. For purposes of this invention, the term ethanol is used interchangeably with the term “absolute alcohol”. The amount of, for example, ethanol in a particular formulation will vary based on the route of delivery of the intended formulation and the solubility of the cannabinoid. The amount of ethanol in the formulations of the present invention can range from about 15 to about 90%; from about 15 to about 65%; and about 15 to about 50% by weight.

In certain preferred embodiments, polyethylene glycol is used as a portion of the organic solvent for the cannabinoid, more preferably a low molecular weight polyethylene glycol is used, most preferably polyethylene glycol 400.

In certain embodiments, the polyethylene glycol comprises from about 1% to about 40% by weight of the non-aqueous cannabinoid formulation; from about 1% to about 30% by weight of the non-aqueous cannabinoid formulation; from about 1% to about 25% by weight of the non-aqueous cannabinoid formulation; more preferably from about 5% to about 30% by weight of the non-aqueous cannabinoid formulation and most preferably from about 5% to about 25% by weight of the non-aqueous cannabinoid formulation by weight.

In certain embodiments, the formulation contains from about 0.1% to about 30% by weight propylene glycol; from about 1% to about 30% by weight propylene glycol; from about 0.1% to about 30% by weight propylene glycol; from about 1% to about 25% by weight propylene glycol; more preferably from about 5% to about 10% of the formulation.

### Solubilizing Agents

In certain embodiments of the invention further solubilizing agents are included in the formulation. Exemplary solubilizing agents include Capryol 90; Cremophor RH40; Labrafil M 1944 CS; Labrafil M 2125 CS; Lauroglycol 90; PEG MW>4000; Plurol Oleique CC 497; poloxamer 124; poloxamer 188; Softigen 701; Softigen 767; Tagat TO; Tween 80; triacetin; triethylcitrate; tributylcitrate; acetyl triethylcitrate; acetyl tributyl citrate; ethyl oleate; ethyl caprylate; ethyl butyrate; triacetin; 2-pyrrolidone; 2-piperidone; N-methylpyrrolidone; M-ethylpyrrolidone; N-hydroxyethyl pyrrolidone; N-octylpyrrolidone; N-laurylpyrrolidone; dimethylacetamide; Miglyol, lanolin, petrolatum, mineral oil and mixtures thereof. The formulations of the present invention may comprise a solubilizing agent from about 0.1% to about 100% of the inactive ingredients; from about 5 to about 75%; or from about 25 to about 50% by weight.

Other components such as preservatives, antioxidants, surfactants, absorption enhancers, viscosity modifiers, film forming polymers, bulking agents, diluents, coloring agents, flavoring agents, pH modifiers, sweeteners or taste-masking agents may also be incorporated into any of the compositions described as part of the invention. The amount of each of these components which may be used will be optimized for each formulation, in order to obtain a stable product (dosage form) having the desired shelf-life. Generally speaking, in embodiments in which these components are included, suitable formulations may include from about 0.001% to about 20% w/w of a pharmaceutically acceptable preservative, antioxidant, surfactant, absorption enhancer, viscosity modifier, film forming polymer, bulking agent, diluent, coloring agent, flavoring agent, pH modifier, sweetener or taste-masking agent.

### Stabilizers

In certain preferred embodiments, the formulation contains amounts of one or more pharmaceutically acceptable stabilizer, such as anti-oxidants, in an amount effective to stabilize the cannabinoid contained therein such that the cannabinoid does not degrade to an unacceptable extent and the formulation is deemed stable as per the ICH guidance for two-year expiration dating when placed under storage conditions selected from (i) 25° C./60% relative humidity (RH) for 12 months; (ii) 30° C./60% relative humidity (RH) for 6 months; (iii) 40° C./60% relative humidity (RH) for 6 months; and (iv) any combination thereof.

In further embodiments of the invention, an effective stabilizing amount of one or more pharmaceutically acceptable stabilizer such as an anti-oxidants is added to the formulation. The term “anti-oxidant” is used herein to describe any compound which is oxidized more easily than the cannabinoid compounds included in the dosage forms of the present invention. Any of the known anti-oxidants may be used, including but not limited to anti-oxidants such as butyl hydroxyl anisole (BHA), butyl hydroxyl toluene (BHT), propyl gallate, lecithin, Vitamin E tocopherol, sesamin, sesamol, sesamolin, alpha tocopherol, ascorbic acid, ascorbyl palmitate, fumaric acid, malic acid, sodium ascorbate and sodium metabisulphite, as well as chelating agents such as disodium EDTA, may also be used to stabilize the cannabinoid formulations of the present invention.

The preparation may also contain anti-oxidant synergists to prevent oxidative degradation. Any of the known anti-oxidant synergists may also be used in effective amounts, for example disodium edetate.

The amount of stabilizer, such as an anti-oxidant, which may be used will be optimized for each formulation, in order to obtain a stable product (dosage form) having the desired shelf-life. Generally speaking, in embodiments in which an anti-oxidant is included, suitable formulations may include from about 0.001% to about 20% w/w of a pharmaceutically acceptable anti-oxidant(s). For example, in certain preferred embodiments, the amount of lecithin included in the cannabinoid dosage form is in the range from about 0.1 to about 10% w/w, and in certain embodiments more preferably from about 0.3% to about 8.25% w/w. In other preferred embodiments, the amount of L-ascorbic acid-6-palmitate is from about 0.001 to about 1%, w/w, and in certain embodiments more preferably in the range from about 0.01% to about 0.1% w/w. The anti-oxidant preferably prevents the formation of degradants in the dosage form such as those mentioned above, namely delta-8 tetrahydrocannabinol (D8THC), cannabinol (CBN), or cannabidiol (CBD), to unacceptable levels (e.g., as previously specified herein).

### Buffers

In addition the formulations may additionally include physiologically acceptable components such as sodium chloride and like materials conventionally used to achieve isotonicity with typical body fluids based on the intended route of administration, e.g., the eye or intravenously. Agents which buffer the pH to maintain a physiologically compatible pH range for the intended route of administration and to enhance the solubility and stability of the active agent present, and the like may also be included in certain embodiments of the present invention.

Suitable buffers include, but are not limited to acetate, bicarbonate, citrate, phosphate, pharmaceutically acceptable salts thereof and combinations or mixtures thereof. When one or more buffers are utilized in the formulations of the invention, they may be combined, e.g., with a pharmaceutically acceptable vehicle and may be present in the final formulation, e.g., in an amount ranging from about 0.1% to about 20%, more preferably from about 0.5% to about 10%. In certain embodiments of the present invention, the amount of buffer included in the gel formulations is preferably an amount such that the pH of the gel formulation does not interfere with the body's natural buffering system causing pain. Therefore, from about 5 mM to about 200 mM concentration of a buffer may be present in the formulations. In certain preferred embodiments, from about a 20 mM to about a 100 mM concentration of a buffer is present. The concentration of buffer is such that a pH of the formulation is from about 5 to about 10; preferably from about 6 to about 8; more preferably from about 6.5 to about 7.5 and most preferably about 7.

In certain other embodiments, the formulations may be isotonic. Isotonic formulations may be provided by the addition of a tonicity agent. Suitable tonicity agents include, but are not limited to any pharmaceutically acceptable sugar, salt or any combinations or mixtures thereof, such as, but not limited to dextrose and sodium chloride. The tonicity agents may be present in an amount from about 100 mOsm/kg to about 500 mOsm/kg. In certain preferred embodiments, the tonicity agent is present in an amount from about 200 mOsm/kg to about 400 mOsm/kg and more preferably from about 280 mOsm/kg to about 320 mOsm/kg.

### Viscosity Modifiers

In further embodiments, the invention is directed to formulations that further contain viscosity modifiers including, for example, cellulose or cellulose derivatives such as ethylcellulose, hydroxyethylcellulose, hydroxypropylcellulose, caboxymethylcellulose, sodium hydroxypropylmethylcellulose, methylcellulose, methylethylcellulose, sodium carboxymethylcellulose, Aerosil, cetostearyl alcohol, Gelucires 33/01, 39/01 and 43/01, glyceryl behenate, glyceryl palmitostearate, Softisans 100, 142, 378 and 649, stearyl alcohol carbomer, xanthan gum, maltodextrin, acacia, tragacanth, povidone and polyvinyl alcohol.

### Absorption Enhancers

Absorption enhancers for use in accordance with certain embodiments of the present invention include, for example, Gelucire 44/14; Gelucire 50/13; Tagat TO; Tween 80; isopropyl myristate, polysorbates, sorbitan esters, poloxamer block copolymers, PEG-35 castor oil, PEG-40 hydrogenated castor oil, caprylocaproyl macrogol-8 glycerides, PEG-8 caprylic/capric glycerides, sodium lauryl sulfate, dioctyl sulfosuccinate, polyethylene lauryl ether, ethoxydiglycol, propylene glycol mono-di-caprylate, glycerol monocaprylate, glyceryl fatty acids (C8-C18) ethoxylated, oleic acid, linoleic acid, glyceryl caprylate/caprate, glyceryl monooleate, glyceryl monolaurate, caprylic/capric triglycerides, ethoxylated nonylphenols, PEG-(8-50) stearates, olive oil PEG-6 esters, triolein PEG-6 esters, lecithin, d-alpha tocopheryl polyethylene glycol 1000 succinate, polycarbonate, sodium glycocholate, sodium taurocholate, cyclodextrins, citric acid, sodium citrate, triacetin, combinations thereof, and the like. In certain preferred embodiments, the absorption enhancer is triacetin. In certain preferred embodiments wherein an absorption enhancer is included in the formulation, the absorption enhancer is included in an amount of from about 0.001% to about 10% by weight of the formulation, preferably in an amount of about 0.01% to about 5% by weight of the formulation.

### Sweetening Agents

In further embodiments, the present invention may comprise a sweetening agent. Suitable sweetening agents include, but are not limited to, sucralose, sucrose, aspartame, saccharin, dextrose, mannitol, xylitol, ethyl maltol, saccharin sodium, dipotassium glycyrrhizinate, stevia, thaumatin, acesulfame K, and combinations thereof. If the formulations contain a sweetener, the formulations preferably contain from about 0.001 to about 1% sweetener.

### Oral Formulations

In further embodiments, the present invention is formulated into a stable non-aqueous cannabinoid formulation by adding, for example, a sweetening agent, taste-masking agent, flavoring agent, coloring agent, viscosity modifying agent or any combinations thereof.

In accordance with non-aqueous oral formulations of the current invention where the cannabinoid is dronabinol, the dronabinol concentration is from about 0.05 mg/ml to about 100 mg/ml; preferably from about 0.5 mg/ml to about 10 mg/ml; and more preferably about 1 mg/ml. The dose of dronabinol provided by the oral formulations is for example from about 2.5 mg to about 50 mg dronabinol.

In certain embodiments the invention is directed to stable non-aqueous cannabinoid formulations for oral administration that contains sucrose, fructose, sorbitol, xylitol, saccharin, saccharin sodium or combinations thereof as a sweetening agent.

One of skill will readily appreciate that the stable non-aqueous cannabinoid oral liquid formulations of the present invention can be incorporated into any pharmaceutically acceptable single-dose or multi-dose container made from any pharmaceutically acceptable material, (e.g., glass or plastic) to allow for oral dosing of the formulation.

### Pharmaceutical Dosage Forms

The compounds and compositions of the present invention can be processed by agglomeration, air suspension chilling, air suspension drying, balling, coacervation, coating, comminution, compression, cryopelletization, encapsulation, extrusion, wet granulation, dry granulation, homogenization, inclusion complexation, lyophilization, melting, microencapsulation, mixing, molding, pan coating, solvent dehydration, sonication, spheronization, spray chilling, spray congealing, spray drying, or other processes known in the art. The compositions can be provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a buccal or sublingual solid, a granule, a film, a sprinkle, a pellet, a bead, a pill, a powder, a triturate, a platelet, a strip or a sachet. Compositions can also be administered as a “dry syrup”, where the finished dosage form is placed directly on the tongue and swallowed or followed with a drink or beverage. These forms are well known in the art and are packaged appropriately. The compositions can be formulated for oral, nasal, buccal, ocular, urethral, transmucosal, vaginal, topical or rectal delivery.

The pharmaceutical composition can be coated with one or more enteric coatings, seal coatings, film coatings, barrier coatings, compress coatings, fast disintegrating coatings, or enzyme degradable coatings. Multiple coatings can be applied for desired performance. Further, the dosage form can be designed for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The definitions of these terms are known to those skilled in the art. In addition, the dosage form release profile can be affected by a polymeric matrix composition, a coated matrix composition, a multiparticulate composition, a coated multiparticulate composition, an ion-exchange resin-based composition, an osmosis-based composition, or a biodegradable polymeric composition. Without wishing to be bound by theory, it is believed that the release may be effected through favorable diffusion, dissolution, erosion, ion-exchange, osmosis or combinations thereof.

When formulated as a capsule, the capsule can be a hard or soft gelatin capsule, a starch capsule, or a cellulosic capsule. Although not limited to capsules, such dosage forms can further be coated with, for example, a seal coating, an enteric coating, an extended release coating, or a targeted delayed release coating. These various coatings are known in the art, but for clarity, the following brief descriptions are provided: seal coating, or coating with isolation layers: Thin layers of up to 20 microns in thickness can be applied for variety of reasons, including for particle porosity reduction, to reduce dust, for chemical protection, to mask taste, to reduce odor, to minimize gastrointestinal irritation, etc. The isolating effect is proportional to the thickness of the coating. Water soluble cellulose ethers are preferred for this application. HPMC and ethyl cellulose in combination, or Eudragit E100, may be particularly suitable for taste masking applications. Traditional enteric coating materials listed elsewhere can also be applied to form an isolating layer.

Extended release coatings are designed to effect delivery over an extended period of time. The extended release coating is a pH-independent coating formed of, for example, ethyl cellulose, hydroxypropyl cellulose, methylcellulose, hydroxymethyl cellulose, hydroxyethyl cellulose, acrylic esters, or sodium carboxymethyl cellulose. Various extended release dosage forms can be readily designed by one skilled in art to achieve delivery to both the small and large intestines, to only the small intestine, or to only the large intestine, depending upon the choice of coating materials and/or coating thickness.

Enteric coatings are mixtures of pharmaceutically acceptable excipients which are applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Additional additives and their levels, and selection of a primary coating material or materials will depend on the following properties: 1. resistance to dissolution and disintegration in the stomach; 2. impermeability to gastric fluids and drug/carrier/enzyme while in the stomach; 3. ability to dissolve or disintegrate rapidly at the target intestine site; 4. physical and chemical stability during storage; 5. non-toxicity; 6. easy application as a coating (substrate friendly); and 7. economical practicality.

Dosage forms of the compositions of the present invention can also be formulated as enteric coated delayed release oral dosage forms, i.e., as an oral dosage form of a pharmaceutical composition as described herein which utilizes an enteric coating to affect release in the lower gastrointestinal tract. The enteric coated dosage form may be a compressed or molded or extruded tablet/mold (coated or uncoated) containing granules, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

Delayed release generally refers to the delivery so that the release can be accomplished at some generally predictable location in the lower intestinal tract more distal to that which would have been accomplished if there had been no delayed release alterations. The preferred method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. Polymers for use in the present invention are anionic carboxylic polymers.

Shellac, also called purified lac, a refined product obtained from the, resinous secretion of an insect. This coating dissolves in media of pH>7. Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

In carrying out the method of the present invention, the combination of the invention may be administered to mammalian species, such as dogs, cats, humans, etc. and as such may be incorporated in a conventional systemic dosage form, such as a tablet, capsule, elixir or injectable. The above dosage forms will also include the necessary carrier material, excipient, lubricant, buffer, antibacterial, bulking agent (such as mannitol), anti-oxidants (ascorbic acid of sodium bisulfite) or the like.

The dose administered must be carefully adjusted according to age, weight and condition of the patient, as well as the route of administration, dosage form and regimen and the desired result.

The pharmaceutical compositions of the invention may be administered in the dosage forms in single or divided doses of one to four times daily. It may be advisable to start a patient on a low dose combination and work up gradually to a high dose combination.

Liquid formulations can be prepared by dissolving or suspending one or the combination of active substances in a conventional liquid vehicle acceptable for pharmaceutical administration so as to provide the desired dosage in one to four teaspoonful.

Dosage forms can be administered to the patient on a regimen of, for example, one, two, three, four, five, six, or other doses per day

In order to more finely regulate the dosage schedule, the active substances may be administered separately in individual dosage units at the same time or carefully coordinated times. Since blood levels are built up and maintained by a regulated schedule of administration, the same result is achieved by the simultaneous presence of the two substances. The respective substances can be individually formulated in separate unit dosage forms in a manner similar to that described above.

In formulating the compositions, the active substances, in the amounts described above, may be compounded according to accepted pharmaceutical practice with a physiologically acceptable vehicle, carrier, excipient, binder, preservative, stabilizer, flavor, etc., in the particular type of unit dosage form.

Illustrative of the adjuvants which may be incorporated in the dosage form are the following: a binder such as gum tragacanth, acacia, corn starch or gelatin; an excipient such as dicalcium phosphate or cellulose; a disintegrating agent such as corn starch, potato starch, alginic acid or the like; a lubricant such as stearic acid or magnesium stearate; a sweetening agent such as sucrose, aspartame, lactose or saccharin; a flavoring agent such as orange, peppermint, oil of wintergreen or cherry. When the dosage unit form is a capsule, it may contain in addition to materials of the above type a liquid carrier such as a fatty oil. Various other materials may be present as coatings or to otherwise modify the physical form of the dosage unit. For instance, capsules may be coated with shellac, sugar or both. A syrup of elixir may contain the active compound, water, alcohol or the like as the carrier, glycerol as solubilizer, sucrose as sweetening agent, methyl and propyl parabens as preservatives, a dye and a flavoring such as cherry or orange.

The liquid formulations useful herein may comprise a solvent, solution, suspension, microsuspension, nanosuspension, emulsion, microemulsion, gel or even a melt containing the active component or components. In some embodiments the nanoparticles, nanofibers, or nanofibrils may be in the form of, or within or on, granules, powders, suspensions, solutions, dissolvable films, mats, webs, tablets, or releasable forms particularly releasable dosage forms. Other particular useful forms are concentrates to which a diluting liquid is added prior to use. The product may also be sprayed onto the inner surface of a container to which a liquid is added later prior to use and the nanoparticles, nanofibers, or nanofibrils, are released into the liquid. Pharmaceutical compositions of the present invention can include nanoparticles, composite nanoparticles, nanosuspension, or nanocapsules of the present invention.

A dose may be administered in a single dosage form or in multiple dosage forms. When multiple dosage forms are used the amount of compound contained within each dosage form may be the same or different. The amount of a composition of the invention contained in a dose may depend on the route of administration and whether the disease in a patient is effectively treated by acute, chronic, or a combination of acute and chronic administration.

In certain embodiments an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, a composition of the invention may exhibit a high therapeutic index. The data obtained from cell culture assays and animal studies may be used in formulating a dosage range that is not toxic for use in, for example, humans. A dose of a composition of the invention provided by the present disclosure may be within a range of circulating concentrations in for example the blood, plasma, or central nervous system, that include the effective dose and that exhibits little or no toxicity. A dose may vary within this range depending upon the dosage form employed and the route of administration utilized. In certain embodiments, an escalating dose may be administered.

### Packaging/Treatment Kits

The present invention relates to a kit for conveniently and effectively carrying out the methods in accordance with the present invention. Such kits may be suited for the delivery of solid oral forms such capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging pharmaceutical unit dosage forms. If desired, the blister can be in the form of a childproof blister, i.e., a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as an AM dose is packaged with a “mid day” and a PM dose.; or an AM dose is packaged with a PM dose. Alternatively, placebo dosages, or vitamin or dietary supplements, either in a form similar to or distinct from the pharmaceutical active dosages, can be included.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the pharmaceuticals of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the invention are contained in-between a card and a clear PVC. The PVC can be transparent so the item (pill, tablet, geltab, etc.) can be seen and examined easily; and in one aspect, can be vacuum-formed around a mold so it can contain the item snugly and have room to be opened upon purchase. In one aspect, the card is brightly colored and designed depending on the item (pill, tablet, geltab, etc.) inside, and the

PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a pharmaceutical combination of the invention), and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or large.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

The invention will be illustrated in more detail with reference to the following Examples, but it should be understood that the present invention is not deemed to be limited thereto.

### EXAMPLES

### Manufacturing Procedure:

1. Dissolve Dronabinol in Dehydrated Alcohol and mix well under continuous argon gas purging.

2. Add remaining ingredients to Dronabinol solution one after another and mix well until get the homogeneous clear solution.

3. Fill the clear solution in suitable HDPE or glass bottle under inert gas.

While the invention has been described in detail and with reference to specific examples thereof, it will be apparent to one skilled in the art that various changes and modifications can be made therein without departing from the spirit and scope thereof.

What is claimed is:

1. An oral non-aqueous formulation comprising:
at least one active cannabinoid;
at least one organic solvent selected from the group consisting of dehydrated alcohol, polyhydric alcohols, polyethylene glycol and propylene glycol, and mixtures thereof;
at least one absorption modifier selected from the group consisting mono-di glycerides, esterified polyalkylene polyols, polyoxyethylene glycolated oils, polyoxyethylene glycolated castor oil, and combinations thereof;
at least one stability enhancing agent selected from the group consisting of tocopherol, butylated hydroxytoluene, butylated hydroxyl anisole, and combinations thereof;
at least one sweetening agents selected from the group consisting of Sucralose, Saccharin Sodium, ethyl maltol, and other flavoring agents.

2. The oral non-aqueous formulation of claim 1 wherein the active cannabinoid is Dronabinol or Δ⁹-Tetrahydrocannabinol.

3. The oral nonaqueous formulation of claim 1 wherein the composition comprises a mixture of solvents and the mixture of organic solvents constitutes 50-95% w/w of the formulation.

4. The oral non-aqueous formulation of claim 1 wherein the organic solvent is dehydrated alcohol and constitutes 15-50% w/w of the formulation.

5. The oral non-aqueous formulation of claim 1 wherein the organic solvent is a polyhydric alcohol selected from the group consisting of sorbitol and glycerol, and constitutes 0-30% w/w of the formulation.

6. The oral nonaqueous formulation of claim 1 wherein the organic solvent is polyethylene glycol which is of a lower molecular weight ranging from 100-800 g/mol and constitutes 5-60% w/w of the entire formulation.

7. The oral non-aqueous formulation of claim 1 wherein the organic solvent is propylene glycol and constitutes 5-45% w/w of the entire formulation.

8. The oral nonaqueous formulation of claim 1 wherein the absorption modulator is polyoxyethylene glycolated castor oil selected from the group consisting of polyoxy 40 hydrogenated castor oil and constitutes 20-40% w/w of the entire formulation.

9. The oral non-aqueous formulation of claim 1 wherein the absorption modulator is a mixture of polyoxyethylene glycolated castor oils and PEGlyated glycerides of fatty acids, and comprises of 0-50% w/w of the entire formulation.

10. The oral non-aqueous formulation of claim 9 wherein the absorption modulator comprises a mixture of polyoxyethylene glycolated castor oil which is polyoxy 40 hydrogenated castor oil, and the PEGlyated glycerides of fatty acid is −8 caprylic/capric glycerides (Labrafil™ or Labrasol™).

11. The oral non-aqueous formulation of claim 1 wherein the stability enhancing agent is selected from the group consisting of Tocopherol, BHA, BHT and mixtures thereof, in which comprises 0-2% w/w of the entire formulation.

12. The oral non-aqueous formulation of claim 1 wherein the sweetening agent is selected from the group consisting of sucralose, saccharin sodium, ethyl maltol, and mixtures thereof and constitutes of about 0-10% w/w of the formulation.

13. A method of treating a patient for a condition selected from the group consisting of pain, emesis, and anorexia comprising:
selecting a patient in need of such treatment;
administering the composition of claim 1,
wherein the patient is treated.
